(19) United States
(12) Patent Application Publication
(10) Pub. No.: US 2025/0325329 A1
(43) Pub. Date: Oct. 23, 2025

(54) Calibration Block For Calibrating A Surgical Tool

(71) Applicant: Stryker Corporation, Portage, MI (US)

(72) Inventor: Caleb Gossens, Ravenna, MI (US)

(73) Assignee: Stryker Corporation, Portage, MI (US)

(21) Appl. No.: 18/870,715

(22) PCT Filed: Jun. 2, 2023
(86) PCT No.: PCT/US2023/024303

§ 371 Date: Dec. 2, 2024

Related U.S. Application Data
(60) Provisional application No. 63/365,744, filed on Jun. 2, 2022.

(51) Int. Cl. A61B 34/20

(52) U.S. Cl. CPC A61B 34/20; A61B 2034/2051; A61B 2034/2055; A61B 2034/207

(57) ABSTRACT

A calibration block for calibrating a hand-held surgical tool coupled to an end effector is provided. The calibration block includes a handle extending along a handle axis and a frame coupled to the handle. The frame includes a first and second frame member each defining a plurality of calibration features configured to receive or be engaged by the end effector. A calibration feature of the first frame member extends from the first frame member in a first direction, and a calibration feature of the second frame member extends from the second frame member in a second direction, the first direction being different from the second direction. The calibration block also includes at least three fiducial markers coupled to the frame, wherein the handle extends in a direction from the frame, and the at least three fiducial markers extend in an opposing direction from the frame.

### CROSS-REFERENCE TO RELATED APPLICATIONS

The present application claims priority to and all the benefits of U.S. Provisional Patent Application No. 63/365,744, filed on Jun. 2, 2022, the entire contents of which are expressly incorporated herein by reference.

### TECHNICAL FIELD

The present disclosure relates generally to devices for use with surgical navigation systems. In particular, the present disclosure relates to devices for calibrating a hand-held surgical tool of a surgical navigation system.

### BACKGROUND

Surgical navigation systems may include hand-held surgical tools for performing a surgical procedure. The hand-held surgical tool requires calibration in order for the surgical navigation system to determine properties of the hand-held surgical tool, such as a type, a shape, and a size of the hand-held surgical tool.

A variety of devices may be used to calibrate a hand-held surgical tool of a surgical navigation system. Typically, these devices include calibration features, which interact with the hand-held surgical tool to calibrate the hand-held surgical tool. However, these devices may be difficult to manipulate during calibration of the hand-held surgical tool and the calibration features may be difficult to access.

As such, there remains a need in the art to provide a device for calibrating a hand-held surgical tool of a surgical navigation system in a manner that is easily manipulable and facilitates access to the calibration features of the calibration device.

### SUMMARY

According to a first aspect, a calibration block for calibrating a hand-held surgical tool coupled to an end effector is provided. The calibration block comprises: a handle extending along a handle axis; a frame coupled to the handle and surrounding the handle axis, the frame comprising a first frame member and a second frame member each defining a plurality of calibration features, wherein a calibration feature of the plurality of calibration features is configured to receive or be engaged by the end effector, wherein a calibration feature of the first frame member is oriented in a first direction, and wherein a calibration feature of the second frame member is oriented in a second direction, the first direction being different from the second direction; and at least three fiducial markers coupled to the frame; wherein the handle extends in a third direction from the frame, and wherein the at least three fiducial markers extend in a fourth direction from the frame, the fourth direction being opposite from the third direction.

For any of the above aspects, any one or more of the following implementations are contemplated, individually or in combination:

In one implementation, the plurality of calibration features includes an aperture and/or a divot. In one implementation, the plurality of calibration features includes at least one of a boss, a groove, a protrusion, and a laser marking.

In one implementation, the frame further comprises a third frame member defining a calibration feature. In one implementation, the calibration feature of the third frame member includes a conical divot.

In one implementation, the frame further comprises a third frame member and a fourth frame member. In one implementation, the first frame member is coupled to the second frame member, wherein the second frame member is coupled to the third frame member, wherein the third frame member is coupled to the fourth frame member, and wherein the fourth frame member is coupled to the first frame member. In one implementation, the first frame member, the second frame member, the third frame member, and the fourth frame member define an opening, the opening being intersected by the handle axis. In one implementation, at least two of the first frame member, the second frame member, the third frame member, and the fourth frame member have different lengths. In one implementation, a distance from the handle axis to one of the first frame member, the second frame member, the third frame member, and the fourth frame member is equal to a distance from the handle axis to another one of the first frame member, the second frame member, the third frame member, and the fourth frame member.

In one implementation, the handle includes a handle surface including a roughness profile having an arithmetical mean height (Ra) greater than 0.01 micrometers and less than 3.0 micrometers.

In one implementation, the handle includes a first end at a first location along the handle axis and a second end at a second location along the handle axis. In one implementation, the frame is coupled to the handle proximate the first end of the handle. In one implementation, the second end of the handle includes a rounded profile. In one implementation, the handle includes one or more grooves extending parallel to the handle axis.

In one implementation, at least three fiducial markers are removably coupled to the frame.

In one implementation, the first frame member includes a first frame member surface being parallel to the handle axis and a second frame member surface being oriented relative to the first frame member surface at an angle between 15 and 75 degrees.

Any of the above implementations can be utilized individually or in combination with any part of any of the above aspects.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of a calibration block.

FIG. 2 is a top diagrammatic view of the calibration block of FIG. 1.

FIGS. 3A-3D are side views of the calibration block of FIG. 1.

FIG. 4 is a perspective view of the calibration block of FIG. 1, wherein

fiducial markers are removably coupled to the calibration block.

FIGS. 5A-5D are side views of alternate examples of a calibration block.

FIG. 6 is a perspective view of the calibration block of FIG. 1, wherein the calibration block is manipulated by a user.

### DETAILED DESCRIPTION

### I. Overview of the Calibration Block

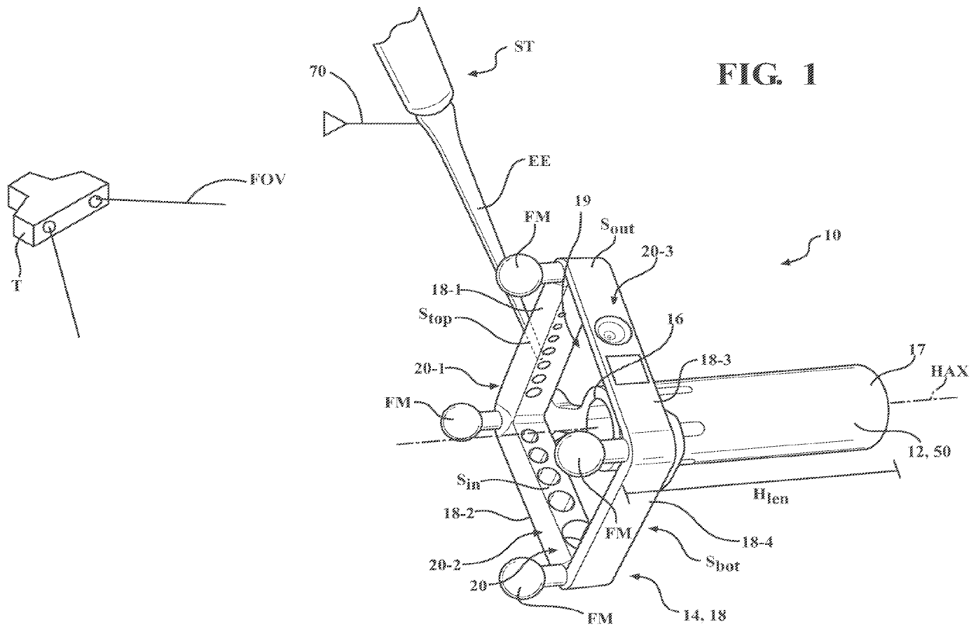

A calibration block 10 for calibrating a hand-held surgical tool ST coupled to an end effector EE is shown in FIG. 1. The calibration block 10 and the hand-held surgical tool ST may be a part of a surgical navigation system. During calibration of the hand-held surgical tool ST, the surgical navigation system determines properties of the end effector EE, such as a type of an end effector EE, a size of the end effector EE, axis of the end effector EE, and/or a shape of the end effector EE.

Specifically, the surgical navigation system may track a position and/or orientation of the calibration block 10 and a position and/or orientation of the surgical tool ST. As will be described in greater detail below, the calibration block 10 may include fiducial markers FM that may be tracked by the surgical navigation system to determine a position and/or orientation of the calibration block 10. As shown in FIG. 1, the hand-held surgical tool ST may include a tool tracker 70 that may be tracked by the surgical navigation system to determine a position and/or orientation of the hand-held surgical tool ST. The tool tracker 70 may be similar to as is disclosed in International Patent Publication No. WO 2023/067503 A1, entitled “Attachment and System for Tracking a Surgical Instrument,” and/or U.S Patent Application Publication No. US 2021/0236212 A1, entitled “Tracker for a Surgical Instrument,” the disclosures of each of which are hereby incorporated by reference in their entirety. Other configurations are contemplated. As such, when the calibration block 10 and the hand-held surgical tool ST interact, the surgical navigation system may track the interaction between the calibration block 10 and the hand-held surgical tool ST to determine a type or identity of an end effector EE, an axis of the end effector EE, a size of the end effector EE, and/or a shape of the end effector EE.

### II. Structure of the Calibration Block

As shown, the calibration block includes a handle 12 and a frame 14. The handle 12 extends along a handle axis HAX and the frame 14 surrounds the handle axis HAX. The frame 14 is coupled to the handle 12. Specifically, the handle 12 includes a first end 16 at a first location along the handle axis HAX and a second end 17 at a second location along the handle axis HAX. As shown, the frame 14 is coupled to the handle 12 at the first end 16.

The frame 14 includes at least two frame members 18. In the instance of the calibration block 10 illustrated herein, and as shown in FIG. 1, the frame members 18 includes a first frame member 18-1, a second frame member 18-2, a third frame member 18-3, and a fourth frame member 18-4. However, in other instances, the frame 14 may include two, three, four, five, or any other suitable number of frame members 18.

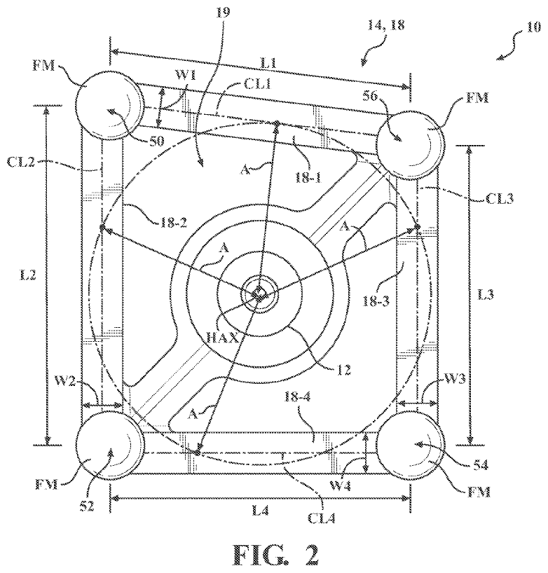

The frame members 18 may be coupled to one another in any suitable fashion. As shown in FIG. 2, the first frame member 18-1 is coupled to the second frame member 18-2, the second frame member 18-2 is coupled to the third frame member 18-3, the third frame member 18-3 is coupled to the fourth frame member 18-4, and the fourth frame member 18-4 is coupled to the first frame member 18-1. Furthermore, the first, second, third, and fourth frame members 18-1, 18-2, 18-3, 18-4 are coupled to one another in a manner so as to define an opening 19. As shown in FIG. 2, the opening 19 includes a quadrilateral cross-sectional shape and, as shown in FIG. 1, the opening 19 is intersected by the handle axis HAX. In instances where the calibration block 10 includes a greater or fewer number of frame members 18, the frame members 18 may be coupled to one another in a different manner such that the opening 19 may include a different cross-sectional shape. For example, the opening 19 may include a rectangular cross-sectional shape, a triangular cross-sectional shape, a round cross-sectional shape, a concave polygonal cross-sectional shape, and/or any other polygonal cross-sectional shape.

Surfaces of the frame members 18 are shown in FIG. 1. Each frame member 18 includes a top surface Sₜₒₚ, a bottom surface S_{bot}, an outer surface Sₒᵤₜ, and an inner surface Sᵢₙ. It should be noted, however, that in other instances, a frame member 18 may include a greater or lesser number of surfaces. For example, in an instance where a frame member 18 includes a pentagonal cross-sectional shape, the frame member 18 may include five surfaces.

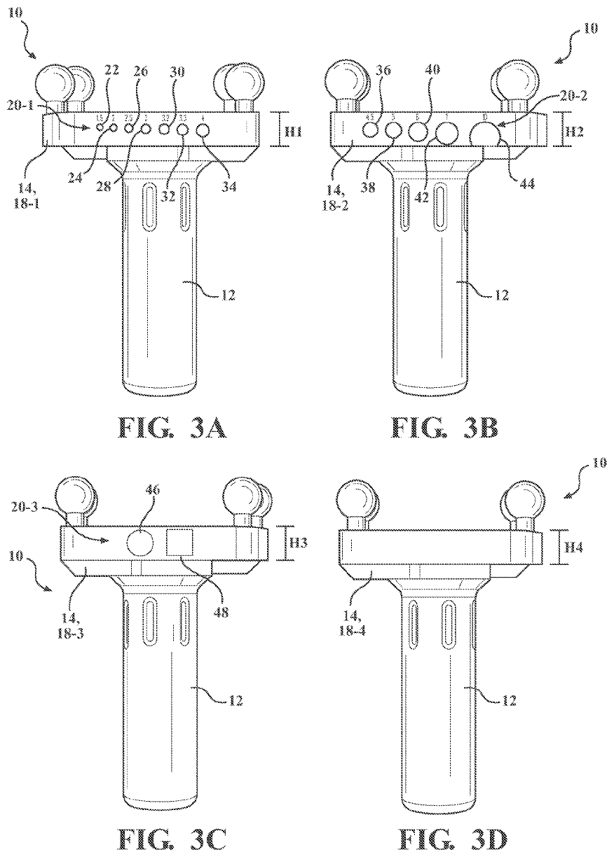

Dimensions of the frame members 18 are shown in FIGS. 2 and 3A-3D. As shown, each frame member 18 includes a length and a width. As shown in FIG. 2, the first frame member 18-1 includes a length L1 and width W1, the second frame member 18-2 includes a length L2 and a width W2, the third member 18-3 includes a length L3 and a width W3, and the fourth frame member 18-4 includes a length L4 and a width W4. As shown in FIGS. 3A-3D, the first frame member 18-1 includes a height H1, the second frame member 18-2 includes a height H2, the third member 18-3 includes a height H3, and the fourth frame member 18-4 includes a height H4.

As shown in FIG. 1, each of the frame members 18 may define a plurality of calibration features 20. In the instance illustrated herein, the first frame member 18-1 defines a plurality of calibration features 20-1, the second frame member 18-2 defines a plurality of calibration features 20-2, and the third frame member 18-3 defines a plurality of calibration features 20-3. As shown in FIG. 3A, the calibration features 20-1 including apertures 22, 24, 26, 28, 30, 32, 34 each having a cylindrical shape and a diameter of 1.5 mm, 2 mm, 2.5 mm, 3 mm, 3.2 mm, 3.5 mm, and 4 mm respectively. As shown in FIG. 3B, the calibration features 20-2 including apertures 36, 38, 40, 42 each having a cylindrical shape and a diameter of 4.5 mm, 5 mm, 6 mm, 7 mm. Additionally, the calibration features 20-2 includes an aperture 44 having a partially cylindrical shape and a diameter of 10 mm. As shown in FIG. 3C, the third frame member 18-3 includes calibration features 20-3 including a conical divot 46 and a rectangular laser marking 48. The conical divot 46 has a diameter of 4.5 mm and an altitude of 2 mm and the rectangular laser marking 48 has a height of 5 mm and a width of 5 mm.

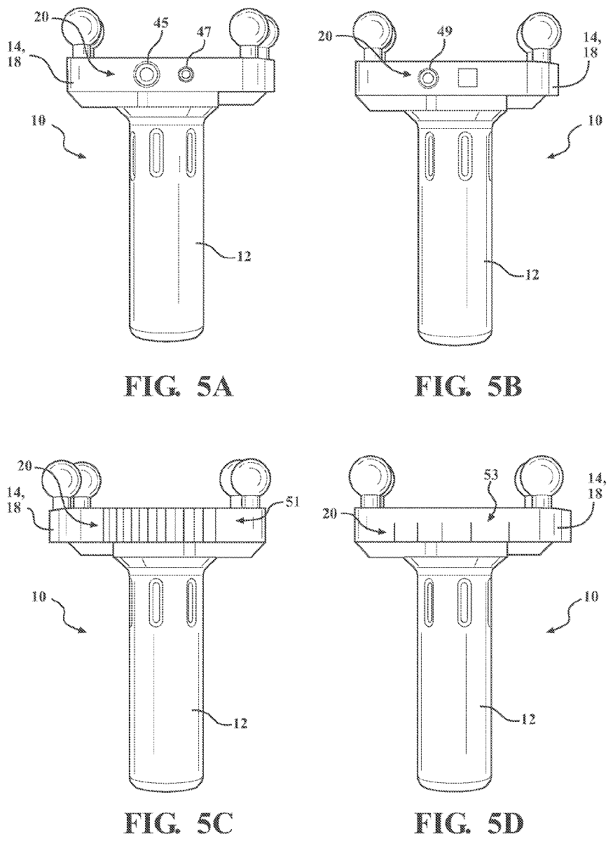

In other instances, the frame members 18 may include a variety of other calibration features 20. For example, as shown in the instance of the calibration block 10 shown in FIG. 5A, the calibration features 20 may include bosses 45, 47. As shown in the instance of FIG. 5B, the calibration features 20 may include a protrusion 49. As shown in the instance of FIG. 5C, the calibration features 20 may include grooves 51. As shown in the instance of FIG. 5D, the calibration features 20 may include linear laser markings 53.

Furthermore, it is contemplated that the frame members 18 may include calibration features 20 not shown in FIGS. 3A-3D and 5A-5D. For instance, the calibration features may include a ridge, a non-linear laser marking, a circular laser marking, and/or an aperture including any suitable geometric shape (e.g. a cuboidal, spherical, pyramidal, ellipsoidal, and/or conical geometric shape) and/or any suitable cross-sectional shape (e.g. a rectangular cross-sectional shape, a triangular cross-sectional shape, a round cross-sectional shape, a concave polygonal cross-sectional shape, and/or any other polygonal cross-sectional shape). Additionally, the calibration features 20 may have any suitable shape and may be of any suitable size.

The frame members 18 may also include any suitable number of calibration features 20. For example, the first frame member 18-1 includes seven calibration features 20-1 (apertures 22, 24, 26, 28, 30, 32, 34), the second frame member 18-2 includes five calibration features 20-2 (apertures 36, 38, 40, 42, 44), and the third frame member 18-3 includes two calibration features 20-3 (conical divot 46 and rectangular laser marking 48). Additionally, the frame members 18 may also optionally omit calibration features 20. For example, in the instance illustrated herein, the fourth frame member 18-4 does not include any calibration features 20.

The calibration features 20 may be oriented in a variety of directions. The orientation of a calibration feature 20 corresponds to an axial tilt angle of the calibration feature 20. For example, in instances where a calibration feature 20 of a frame member 18 is an aperture including a cylindrical shape, the cylindrical aperture may be defined as including an axis parallel to a height of the cylindrical aperture. As follows, the orientation of the cylindrical aperture corresponds to a tilt angle of the axis of the cylindrical aperture relative to the outer or inner surface Sₒᵤₜ, Sᵢₙ of the frame member 18. In instances where a calibration feature 20 of a frame member 18 is a divot including a conical shape, the conical divot may be defined as including an axis extending from a vertex of the conical divot through a center of a base of the conical divot. As follows, the orientation of the conical divot corresponds to a tilt angle of the axis of the conical divot relative to the outer or inner surface Sₒᵤₜ, Sᵢₙ of the frame member 18. It should be noted that, in other instances, the calibration features 20 may include any suitable geometric and/or cross-sectional shape. In such instances, the orientation of the calibration feature corresponds to a tilt angle of an axis of the calibration feature.

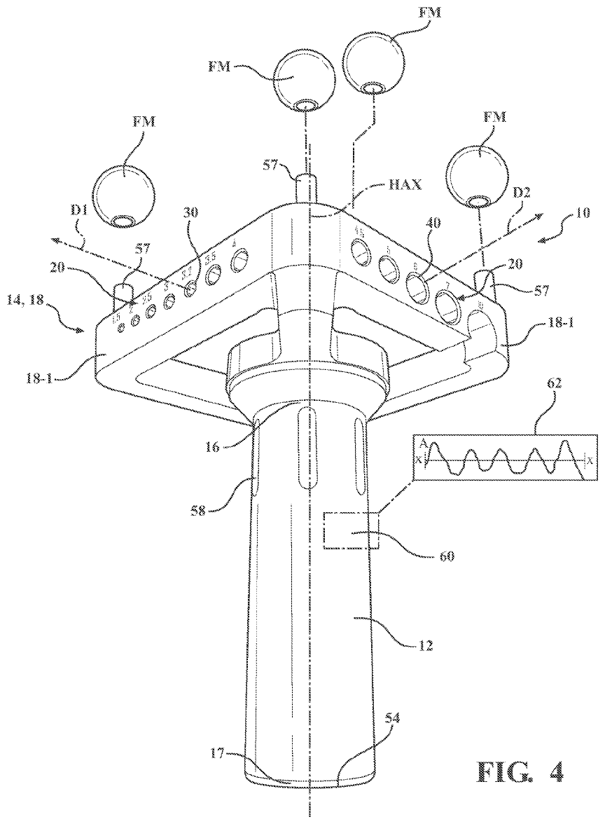

In instances where the calibration block 10 includes the first, second, third, and fourth frame member 18-1, 18-2, 18-3, 18-4, a calibration feature 20 of the first frame member 18-1 may be oriented in a first direction, a calibration feature 20 of the second frame member 18-2 may be oriented in a second direction, a calibration feature 20 of the third frame member 18-3 may be oriented in a third direction, and a calibration feature 20 of the fourth frame member 18-4 be oriented in a fourth direction, the first, second, third, and fourth direction being any suitable direction. Furthermore, in some instances, at least one of the first, second, third, and fourth direction may be different. Specifically, in the instance illustrated herein, and as shown in FIG. 4, the aperture 30 of the first frame member 18-1 is oriented in a first direction D1, and the aperture 40 of the second frame member 18-2 is oriented in a second direction D2 with the first direction D1 being different from the second direction D2.

As previously stated, the calibration block 10 and the hand-held surgical tool ST interact to calibrate the hand-held surgical tool ST. In one instance, the calibration features 20 may be configured to engage the end effector EE to calibrate the hand-held surgical tool ST. For example, the end effector EE may be inserted into an aperture 22, 24, 26, 28, 30, 32, 34, 36, 38, 40, 42, 44 of the first or second frame member 18-1, 18-2, such that the aperture 22, 24, 26, 28, 30, 32, 34, 36, 38, 40, 42, 44 receives the end effector EE, to calibrate the hand-held surgical tool ST. As another example, the end effector EE may be inserted into and rotated within the conical divot 46, such that the conical divot 46 receives the end effector EE, to calibrate the hand-held surgical tool ST. As yet another example, the end effector EE may be inserted into and run along a groove 51, such that the groove 51 receives the end effector EE, to calibrate the hand-held surgical tool ST.

Referring to FIG. 1, the calibration block 10 includes fiducial markers FM. The fiducial markers FM are tracked by the surgical navigation system to determine a position and/or orientation of the calibration block 10. The calibration block 10 may include any suitable number of fiducial markers FM for allowing the surgical navigation system to track the calibration block 10. For example, the calibration block 10 may include at least three fiducial markers FM such that the surgical navigation system may track the calibration block 10 in a three-dimensional plane. In other instances, the calibration block 10 may include a greater number of fiducial markers FM to provide the surgical navigation system with additional information for determining the position and/or orientation of the calibration block 10. In the instance illustrated herein, the calibration block 10 includes four fiducial markers FM.

The fiducial markers FM may be active fiducial markers FM. For example, the fiducial markers FM may include light emitting diodes configured to emit continuous or pulsed light. As another example, the fiducial markers FM may be RF markers configured to broadcast a radiofrequency signal. The fiducial markers FM may also be passive fiducial markers FM. For example, the fiducial markers FM may be reflective markers configured to reflect visible light and/or infrared light. As another example, the fiducial markers FM may include a barcode, such as a QR code, and/or identifying markings.

In the instance illustrated herein, the fiducial markers FM are coupled to the frame 14. Specifically, as shown in FIG. 2, the fiducial markers FM are coupled to the frame 14 at a coupling point 50 between the first frame member 18-1 and the second frame member 18-2, a coupling point 52 between the second frame member 18-2 and the fourth frame member 18-4, a coupling point 54 between the third frame member 18-3 and the fourth frame member 18-4, and a coupling point 56 between the first frame member 18-1 and the third frame member 18-3. In other instances, the fiducial markers FM may be additionally or alternatively coupled to any other portion of the frame 14, such as along a length L1, L2, L3, L4 of a frame member 18. In alternative instances, the fiducial markers FM may be additionally or alternatively coupled to other components of the calibration block 10, such as the handle 12.

The fiducial markers FM may be coupled to the calibration block 10 using any suitable method. In the illustrated instance, and as shown in FIG. 4, each fiducial markers FM may be attached to a mount 57 in order to be coupled to the frame 14. The mounts 57 are posts located at the coupling points 50, 52, 54, 56 to which the fiducial markers FM may be removably attached. In other instances, a fiducial marker FM may be screwed or welded to the calibration block 10. In other instances, the fiducial markers FM may be integral to the calibration block 10.

The calibration block 10 and components thereof, such as the frame 14 and the handle 12, may include any suitable shape.

For example, each frame member 18 of the frame 14 may include any suitable length and width. In the instance illustrated herein, the first, second, and third frame members 18-1, 18-2, 18-3 vary in length, while the third and fourth frame members 18-3, 18-4 are of equivalent length. Specifically, length L1 is 62.4 mm, the length L2 is 55.5 mm, length L3 is 55 mm, and the length L4 is 55 mm. In other instances, at least one of the first, second, third, and fourth frame members 18-1, 18-2, 18-3, 18-4 has a different length. In other instances, the first, second, third, and fourth frame members 18-1, 18-2, 18-3, 18-4 are all equivalent in length. In the instance illustrated herein, the first, second, and third frame members 18-1, 18-2, 18-3, 18-4 are all equivalent in width. In other instances, at least one of the first, second, third, and fourth frame members 18-1, 18-2, 18-3, 18-4 has a different width.

Each frame member 18 may also include any suitable height. For example, in the instance illustrated herein, the first, second, third, and fourth frame members 18-1, 18-2, 18-3, 18-4 are equivalent in height. In other instances, at least one of the first, second, third, and fourth frame members 18-1, 18-2, 18-3, 18-4 has a different height.

Each frame member 18 may also include any suitable geometric shape and any suitable cross-sectional shape. In the instances illustrated herein, each frame member 18 includes a prismatic geometric shape and a quadrilateral cross-section. However, in other instances, a frame member 18 may include a cylindrical, cuboidal, spherical, pyramidal, ellipsoidal, and/or conical geometric shape. Furthermore, a frame member 18 may include a rectangular cross-sectional shape, a triangular cross-sectional shape, a round cross-sectional shape, a concave polygonal cross-sectional shape, and/or any other polygonal cross-sectional shape.

Additionally, the handle 12 may include any suitable length, any suitable geometric shape, and any suitable cross-sectional shape. Referring to FIG. 1, the handle 12 includes a length Hlen extending along the handle axis HAX. In the instance of FIG. 1, the length Hlen is approximately 85.2 cm. However, in other instances, the length Hlen may be longer or shorter than 85.2 cm. Also shown in FIG. 1, the handle 12 includes a cylindrical shape and a circular cross-sectional shape. However, in other instances, the handle 12 may include a cuboidal, spherical, pyramidal, ellipsoidal, and/or conical geometric shape. Furthermore, the handle 12 may include a rectangular cross-sectional shape, a triangular cross-sectional shape, a round cross-sectional shape, a concave polygonal cross-sectional shape, and/or any other polygonal cross-sectional shape.

The calibration block 10 and components thereof may be formed of any suitable material. For example, the calibration block 10 may be formed of a metal (such as aluminum) or metal alloy (such as an aluminum alloy) and/or a polymeric material and/or an elastomeric material. Additionally, components of the calibration block 10, such as the handle 12, the frame 14, and the fiducial markers FM, may be coupled to one another or integrally formed with one another.

### III. Advantageous Features of the Calibration Block

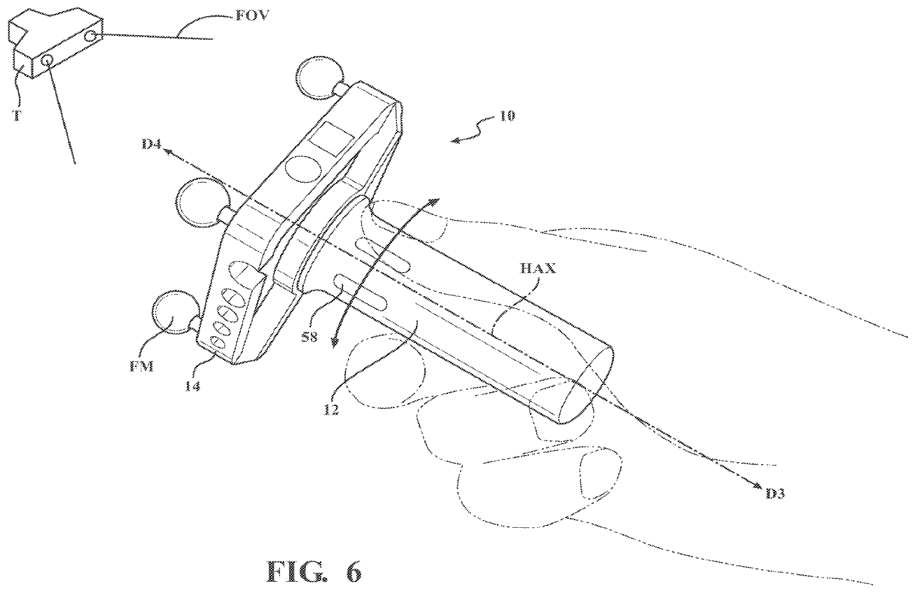

The calibration block 10 provides a user an ergonomic solution to calibrating a hand-held surgical tool. For example, a user may rotate the handle 12 of the calibration block 10 using one hand, as shown in FIG. 6. This allows the user to hold a hand-held surgical tool ST in one hand, while rotating the calibration block 10 with the other hand. In this way, a user may access a calibration feature 20 of any frame member 18 while holding a hand-held surgical tool ST. For example, referring to FIG. 4, a user may rotate the handle 12 until the aperture 30, which extends in the first direction D1, extends toward the user.

The handle 12 may include several features to facilitate rotation of the handle 12 by a user.

The handle 12 may include a finish that increases the friction between a hand of the user and the handle 12, allowing the user to easily grip and rotate the handle 12. In such an instance, the handle 12 may include a handle surface 60 including a roughness profile 62 (both shown in FIG. 4). The roughness profile 62 may be any suitable roughness profile and may be characterized using any suitable parameter, such as an arithmetical mean height (Rₐ), a maximum height (Rz), and/or a mean height of profile elements (R_{c}). For instance, the roughness profile 62 may have an arithmetical mean height (Rₐ) greater than 0.01 micrometers and less than 3.0 micrometers to allow the user to easily grip and rotate the handle 12. In other instances, the roughness profile 62 may vary. For example, the arithmetical mean height (Rₐ) may be greater than 0.01 micrometers and less than 10.0 micrometers, greater than 0.01 micrometers and less than 5.0 micrometers, greater than 1.0 micrometers and less than 5.0 micrometers, greater than 3.0 micrometers and less than 7.0 micrometers, greater than 5.0 micrometers and less than 10.0 micrometers, and/or greater than 0.5 micrometers and less than 2.0 micrometers.

The second end 17 of the handle 12 may include a rounded profile 54, shown in FIG. 4. The rounded profile 54 allows the handle 12 to comfortably rest in a palm of the user while the user rotates the handle 12. For example, the second end 17 of the handle 12 may include a spherical or ellipsoidal profile. In other instances, the handle 12 may include a rounded edge proximate the second end 17. In still other instances, the handle 12 may include a chamfered edge proximate the second end 17.

The handle 12 may include grooves 58, shown in FIG. 4. The grooves 58 provide the user with a manipulable element that the user may contact to more easily rotate the handle 12, as shown in FIG. 6. In the instances illustrated herein, the grooves 58 extend along an axis that is parallel to the handle axis HAX and are disposed proximate the first end 16. In other instances, the handle 12 may include any suitable number of grooves 58, the grooves 58 may extend in any suitable direction, and the grooves 58 may be disposed at any location along the handle 12. For example, in an alternate instance, the handle 12 may include a single groove 58 located at a midpoint along the handle axis HAX between the first end 16 and the second end 17, and the groove 58 may extend at an oblique angle relative to the handle axis HAX. Furthermore, in other instances, the handle 12 may include any suitable manipulable element. For example, the handle 12 may additionally or alternatively include protrusions and/or bosses, which the user may contact to more easily rotate the handle 12.

The fiducial markers FM may be coupled to the calibration block 10 to maintain an ability of the surgical navigation system to track the calibration block 10, while allowing a user to conveniently grip the handle 12. Specifically, referring to FIG. 6, the handle 12 extends in a direction D3 from the frame 14 and the fiducial markers FM extend from the frame 14 in an opposing direction D4. In some instances, the direction D4 may extend away from the user and toward a camera and/or localizer of the surgical navigation system (shown as localizer T with a field of view FOV in FIG. 1). This allows the camera and/or localizer to easily sense the fiducial markers FM and for the surgical navigation system to easily track the calibration block 10 during calibration, while still positioning the calibration features at a convenient location relative to the handle. Furthermore, as the user rotates the handle 12 while using the calibration block 10, the fiducial markers FM continue to extend toward the camera, allowing the camera and/or localizer to continue to sense the fiducial markers FM during use of the calibration block 10 and for the surgical navigation system to continue track the calibration block 10 during calibration.

The frame 14 may include features to facilitate a user's access to a calibration feature 20. For example, the frame members 18 may be coupled such that a distance from the handle axis HAX to one of the frame members 18 is equivalent to a distance from the handle axis HAX to another one of the frame members 18. Specifically, as shown in FIG. 2, a circle with radius A may defined around the handle axis HAX. Additionally, each frame member 18 includes a center line disposed at a center of a width of the frame member, the center line extending parallel to the length of the frame member 18. For example, the first frame member 18-1 includes a center line CL1 disposed at a center of width W1 and extending parallel to length L1, the second frame member 18-2 includes a center line CL2 disposed at a center of width W2 and extending parallel to length L2, the third frame member 18-3 includes a center line CL3 disposed at a center of width W3 and extending parallel to length L3, and fourth frame member 18-4 includes a center line CL4 disposed at a center of width W4 and extending parallel to length L4. Therefore, as shown in FIG. 2, a distance from the handle axis HAX to one of the frame members 18 is equivalent to a distance from the handle axis HAX to another one of the frame members 18 as a distance between a point along the center line CL1, CL2, CL3, CL4 and the handle axis HAX is equivalent to the radius A. In this way, a user of the calibration block 10 may easily rotate the frame 14 by rotating the handle 12 as the frame members 18, and the weight of each frame member 18, are similarly displaced about the handle axis HAX. Additionally, a location of the calibration features 20 with respect to the handle 12 is consistent, regardless of which frame member 18 is facing the user.

It is contemplated that the radius A may be any suitable value. For example, the radius A may be defined as a range of 30 mm-40 mm, 25 mm-35 mm, 35 mm-45 mm, 10 mm-50 mm, 50 mm-100 mm, or 75 mm-150 mm. As follows, the frame members 18 and calibration features 20 may be within a range of 30 mm-40 mm, 25 mm-35 mm, 35 mm-45 mm, 10 mm-50 mm, 50 mm-100 mm, or 75 mm-150 mm from the handle axis HAX.

The calibration block 10 may include several features to facilitate tracking of the calibration block 10 by a surgical navigation system including a camera and/or a localizer, the camera and/or localizer being configured to sense the fiducial markers FM.

For example, referring to FIG. 1, one or more frame members 18 may include one or more angled surfaces to reduce light reflecting back into the camera and/or localizer of the surgical navigation system. For instance, as shown, the outer surface Sₒᵤₜ of the first frame member 18-1 may be parallel to the handle axis HAX and the top surface Sₜₒₚ may be oriented relative to the first frame member surface at an oblique angle. As such, when light is reflected towards the frame 14 and, specifically, towards a frame member 18, the light is reflected away from the camera and/or localizer, allowing for more accurate sensing of the fiducial markers FM by the camera and/or localizer, and for more accurate tracking of the calibration block 10 by the surgical navigation system. In the instance of FIG. 1, the oblique angle is approximately 40 degrees. However, in other instances, the oblique angle may be any suitable angle, such as any angle between 15 and 75 degrees.

### IV. Example Use of the Calibration Block

A user manipulates the hand-held surgical tool ST to interact with the calibration block 10 to determine a type or identity of an end effector EE, an axis of the end effector EE, a size of the end effector EE, and/or a shape of the end effector EE. Additionally, the user may hold the hand-held surgical tool ST in one hand, while rotating the calibration block 10 with the other hand to access a calibration feature 20 of any frame member 18 while holding a hand-held surgical tool ST.

In one example use, the user may manipulate the handle 12 of the calibration block 10 in a first hand while manipulating the calibration block 10 with a second hand to facilitate access to a calibration feature 20 of a frame member 18. For instance, a calibration feature 20 of the first frame member 18-1 may be easily accessible to the user while the calibration block 10 is in a first pose and a second calibration feature 20 of the first frame member 18-1 may be easily accessible to the user while the calibration block 10 is in a second pose. In such an instance, the user may hold the calibration block 10 with a first hand in the first pose such that the at least three fiducial markers FM are within a view of the camera and/or localizer T. While the calibration block 10 is in the first pose, the user may position the hand-held surgical tool ST with a second hand such that at least a portion of the hand-held surgical tool ST, such as the end effector EE, contacts at least one of the calibration features 20 of the first frame member 18-1. The user may then spin the handle 12 of the calibration block 10, within the first hand, to rotate the calibration block 10 from the first pose to the second pose while the fiducial markers FM of the calibration block 10 remain within the view of the camera and/or localizer T. While the calibration block 10 is in the second pose, the user may position the hand-held surgical tool ST with the second hand such that at least a portion of the hand-held surgical tool ST, such as the end effector EE, contacts at least one of the calibration features 20 of the second frame member 18-2 while the calibration block is in the second pose.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A calibration block for calibrating a hand-held surgical tool coupled to an end effector, the calibration block comprising:
a handle extending along a handle axis;
a frame coupled to the handle and surrounding the handle axis, the frame comprising a first frame member and a second frame member each defining a plurality of calibration features, wherein a calibration feature of the plurality of calibration features is configured to receive the end effector or be engaged by the end effector, wherein a calibration feature of the first frame member is oriented in a first direction, and wherein a calibration feature of the second frame member is oriented in a second direction, the first direction being different from the second direction; and
at least three fiducial markers coupled to the frame;
wherein the handle extends in a third direction from the frame, and wherein the at least three fiducial markers extend in a fourth direction from the frame, the fourth direction being opposite from the third direction.

2. The calibration block of any preceding claim, wherein the plurality of calibration features includes an aperture and/or a divot.

3. The calibration block of any preceding claim, wherein the plurality of calibration features includes at least one of a boss, a groove, a protrusion, and a laser marking.

4. The calibration block of any preceding claim, wherein the frame further comprises a third frame member defining a calibration feature.

5. The calibration block of claim 4, wherein the calibration feature of the third frame member includes a conical divot.

6. The calibration block of any preceding claim, wherein the frame further comprises a third frame member and a fourth frame member.

7. The calibration block of claim 6, wherein the first frame member is coupled to the second frame member, wherein the second frame member is coupled to the third frame member, wherein the third frame member is coupled to the fourth frame member, and wherein the fourth frame member is coupled to the first frame member.

8. The calibration block of claim 6, wherein the first frame member, the second frame member, the third frame member, and the fourth frame member define an opening, the opening being intersected by the handle axis.

9. The calibration block of claim 6, wherein at least two of the first frame member, the second frame member, the third frame member, and the fourth frame member have different lengths.

10. The calibration block of claim 6, wherein a distance from the handle axis to one of the first frame member, the second frame member, the third frame member, and the fourth frame member is equal to a distance from the handle axis to another one of the first frame member, the second frame member, the third frame member, and the fourth frame member.

11. The calibration block of any preceding claim, wherein the handle includes a handle surface.

12. The calibration block of any preceding claim, wherein the handle includes a roughness profile having an arithmetical mean height (Rₐ) greater than 0.01 micrometers and less than 3.0 micrometers.

13. The calibration block of any preceding claim, wherein the handle includes a first end at a first location along the handle axis and a second end at a second location along the handle axis.

14. The calibration block of claim 13, wherein the frame is coupled to the handle proximate the first end of the handle.

15. The calibration block of claim 13, wherein the second end of the handle includes a rounded profile.

16. The calibration block of claim 13, wherein the handle includes one or more grooves extending parallel to the handle axis.

17. The calibration block of any preceding claim, wherein the at least three fiducial markers are removably coupled to the frame.

18. The calibration block of any preceding claim, wherein the first frame member includes a first frame member surface being parallel to the handle axis and a second frame member surface being oriented relative to the first frame member surface at an angle between 15 and 75 degrees.

19. A calibration block for calibrating a hand-held surgical tool coupled to an end effector, the calibration block comprising:
a handle;
a frame coupled to the handle, the frame comprising a first frame member and a second frame member each defining a plurality of calibration features, wherein a calibration feature of the plurality of calibration features is configured to be engaged by the end effector, wherein a calibration feature of the first frame member is oriented in a first direction, and wherein a calibration feature of the second frame member is oriented in a second direction, the first direction being different from the second direction; and
at least three fiducial markers coupled to the frame;
wherein the handle extends in a third direction relative to the frame and wherein the at least three fiducial markers extend in a fourth direction relative to the frame, the fourth direction being opposite from the third direction.

20. A method for calibrating a hand-held surgical tool coupled to an end effector, the method comprising:
providing a calibration block, the calibration block including a handle extending along a handle axis, a frame coupled to the handle and surrounding the handle axis, the frame comprising a first frame member and a second frame member each defining a plurality of calibration features, and at least three fiducial markers coupled to the frame;
holding the handle of the calibration block with a first hand of a user in a first pose such that the at least three fiducial markers are within a view of a localizer;
positioning a surgical device with a second hand of the user such that at least a portion of the surgical device contacts at least one of the calibration features of the first frame member while the calibration block is in the first pose;
spinning the handle of the calibration block from the first pose to a second pose within the first hand while the fiducial markers of the calibration block remain within the view of a localizer; and
positioning the surgical device with a second hand of the user such that at least a portion of the surgical device contacts at least one of the calibration features of the second frame member while the calibration block is in the second pose.
